(19) United States
(12) Patent Application Publication
(10) Pub. No.: US 2026/0306539 A1
(43) Pub. Date: Oct. 8, 2026

(54) XENOGENEIC ANTIGEN PRESENTING CELLS AND USES THEREOF

(71) Applicants: David BERGLUND, New York, NY (US); Erik BERGLUND, New York, NY (US)

(72) Inventors: David BERGLUND, New York, NY (US); Erik BERGLUND, New York, NY (US)

(21) Appl. No.: 19/474,082

(22) PCT Filed: Apr. 11, 2024
(86) PCT No.: PCT/US2024/023972

§ 371 Date: Oct. 9, 2025

Related U.S. Application Data
(60) Provisional application No. 63/458,843, filed on Apr. 12, 2023.

(51) Int. Cl. A61K 35/15; A61K 9/00; A61K 45/06; A61P 35/00

(52) U.S. Cl. CPC A61K 35/15; A61K 9/0019; A61K 45/06; A61P 35/00

(57) ABSTRACT

The present disclosure relates to methods for treating a tumor in a subject via administering (e.g, by intratumoral injection) a composition comprising antigen presenting cells obtained from a species that is different than the subject, i.e., xenogeneic antigen presenting cells. The source of such xenogeneic antigen presenting cells could be one or more swine. The present disclosure also relates to methods of treating tumors via administering the xenogeneic antigen presenting cells in combination with other anti-cancer therapies. Furthermore, the present disclosure relates to pharmaceutical compositions comprising xenogeneic antigen presenting cells.

### CROSS-REFERENCES TO RELATED APPLICATIONS

This application claims the benefit of U.S. Provisional Patent Application No. 63/458,843, filed Apr. 12, 2023, which is incorporated herein by reference in its entirety.

### 1. FIELD OF THE DISCLOSURE

The present disclosure is in the field of compositions comprising xenogeneic antigen presenting cells (e.g., from one or more swine), methods of producing such compositions, and uses of such compositions for treating tumors.

### 2. BACKGROUND OF THE DISCLOSURE

Traditional cancer therapies, such as chemotherapy, surgery, radiation, etc. are often inadequate in treating cancer patients, and are usually associated with severe side effects. In this regard, cancer immunotherapy has offered several advantages over traditional cancer therapies. Numerous different immunotherapy approaches have been investigated. One such immunotherapy strategies has involved intratumoral administration of allogeneic inflammatory dendritic cells, typically in combination with another anticancer therapy, for treatment of certain cancers (see, e.g., Jin et al. (2022), Oncoimmunology, 11 (1): e2099642; Fröbom et al. (2020), Cancer Immunol. Immunother., 69 (11): 2393-2401; Karlsson-Parra et al. (2018), Pharm. Res., 35:156). For example, ilixadencel, a human monocyte-derived allogeneic dendritic cell-based product stimulated with potent activators, has been investigated in human clinical trials (see, e.g., Fröbom et al. (2020), Cancer Immunol. Immunother., 69 (11): 2393-2401). There still remains a need for cancer immunotherapies that demonstrate a robust immune response.

Citation of a reference herein shall not be construed as an admission that such is prior art to the present disclosure.

### 3. SUMMARY OF THE DISCLOSURE

In one aspect, provided herein are methods for treating a tumor in a subject in need thereof comprising administering a composition comprising antigen presenting cells to the subject, wherein the antigen presenting cells are obtained from a species that is different than the subject.

In one embodiment, the tumor is a solid tumor, and the administering is into the tumor of the subject via an intratumoral injection.

In one embodiment, the antigen presenting cells are obtained from a species that is a swine. In one embodiment, the antigen presenting cells are obtained from a species that is a miniature swine. In one embodiment, the subject is a human. In one embodiment, the swine is an alpha-1,3 galactosyltransferase-deficient swine. In one embodiment, the alpha-1,3 galactosyltransferase-deficient swine is a swine leukocyte antigen (SLA)-inbred swine. In one embodiment, the miniature swine is an alpha-1,3 galactosyltransferase-deficient miniature swine. In one embodiment, the alpha-1,3 galactosyltransferase-deficient miniature swine is a swine leukocyte antigen (SLA)-inbred swine.

In one embodiment, method triggers an immune response specific to the tumor. In one embodiment, the method yields an abscopal effect.

In one embodiment, the tumor is a solid cancerous tumor. In one embodiment, the tumor is selected from the group consisting of sarcomas, carcinomas, lymphomas, breast tumors, prostate tumors, head and neck tumors, glioblastomas, bladder tumors, pancreatic tumors, liver tumors, ovarian tumors, colorectal tumors, pulmonary tumors, cutaneous tumors, lymphoid tumors, gastrointestinal tumors, gastrointestinal stromal tumors, cervical tumors, hepatocellular carcinomas, renal cell carcinomas, melanomas, colorectal carcinomas, esophageal carcinomas, brain tumors, kidney tumors, lung tumors (including non-small cell lung cancer), gastric tumors, bile-duct tumors, uterine tumors, and childhood (pediatric) tumors. In one embodiment, the tumor is resistant to treatment to chemotherapy and/or treatment with an immunotherapy.

In one embodiment, the antigen presenting cells are derived from one or more swine or miniature swine using a leukapheresis procedure, wherein the leukapheresis procedure generates a leukopak containing peripheral blood mononuclear cells, and the leukopak is further fractionated by counterflow elutriation.

In one embodiment, the composition comprising antigen presenting cells is substantially free of pathogens.

In one embodiment, the antigen presenting cells are obtained from swine or miniature swine of different genotypes.

In one embodiment, the composition comprising antigen presenting cells is administered in single or multiple doses. In one embodiment, the composition comprising antigen presenting cells is administered via an intratumoral injection of at least about 1×10⁶ antigen presenting cells per dose. In one embodiment, the composition comprising antigen presenting cells is administered via an intratumoral injection of about 1×10⁶, about 5×10⁶, about 10×10⁶, about 15×10⁶, about 20×10⁶, about 25×10⁶, about 30×10⁶, about 35×10⁶, about 40×10⁶, about 45×10⁶, about 50×10⁶, about 55×10⁶, about 60×10⁶, about 65×10⁶, about 70×10⁶, about 75×10⁶, about 80×10⁶, about 85×10⁶, about 90×10⁶, about 95×10⁶, about 10×10⁷, about 15×10⁷, about 20×10⁷, about 25×10⁷, about 30×10⁷, about 35×10⁷, about 40×10⁷, about 45×10⁷, or about 50×10⁷ antigen presenting cells per dose.

In one embodiment, the antigen presenting cells are substantially mature antigen presenting cells.

In one embodiment, the antigen presenting cells are not activated or stimulated.

In one embodiment, the composition comprises peripheral blood mononuclear cells (PBMC).

In one embodiment, the composition comprises monocytes.

In one embodiment, the composition comprises dendritic cells, macrophages, granulocytes, T-cells, B-cells, and/or NK cells.

In one embodiment, the composition comprises at least about 50%, about 55%, about 60%, about 65%, about 70%, about 75%, about 80%, about 85%, about 90%, about 95%, or greater than about 95% PBMCs, wherein the PBMCs are mature, immature, or a combination of mature and immature PBMCs.

In one embodiment, the composition comprises at least about 50%, about 55%, about 60%, about 65%, about 70%, about 75%, about 80%, about 85%, about 90%, about 95%, or greater than about 95% monocytes, wherein the monocytes are mature, immature, or a combination of mature and immature monocytes.

In one embodiment, the composition comprises a mixture of at least about 50%, about 55%, about 60%, about 65%, about 70%, about 75%, about 80%, about 85%, about 90%, about 95%, or greater than about 95% monocytes and dendritic cells, wherein the monocytes and/or dendritic cells in said mixture may be mature, immature, or a combination of mature and immature monocytes and/or dendritic cells.

In one embodiment, the mixture comprising monocytes and dendritic cells comprises from 10% to 95%, 10% to 90%, 10% to 80%, 10% to 70%, 10% to 60%, 10% to 50%, 10% to 40%, 10% to 30%, 10% to 20%, 20% to 95%, 20% to 90%, 20% to 80%, 20% to 70%, 20% to 60%, 20% to 50%, 20% to 40%, 20% to 30%, 30% to 95%, 30% to 90%, 30% to 80%, 30% to 70%, 30% to 60%, 30% to 50%, 30% to 40%, 40% to 95%, 40% to 90%, 40% to 80%, 40% to 70%, 40% to 60%, 40% to 50%, 50% to 95%, 50% to 90%, 50% to 80%, 50% to 70%, 50% to 60%, 60% to 95%, 60% to 90%, 60% to 80%, 60% to 70%, 70% to 95%, 70% to 90%, 70% to 80%, 80% to 95%, 80% to 90%, 90% to 95%, or greater than 95% monocytes. In one embodiment, the mixture comprising monocytes and dendritic cells comprises from 10% to 30%, 35% to 55%, 60% to 80%, or 85% to 95% monocytes. In one embodiment, the mixture comprises at least about 10%, about 15%, about 20%, about 25%, about 30%, about 35%, about 40%, about 45% about 50%, about 55%, about 60%, about 65%, about 70%, about 75%, about 80%, about 85%, about 90%, about 95%, or greater than about 95% monocytes.

In one embodiment, the mixture comprising monocytes and dendritic cells comprises from 10% to 95%, 10% to 90%, 10% to 80%, 10% to 70%, 10% to 60%, 10% to 50%, 10% to 40%, 10% to 30%, 10% to 20%, 20% to 95%, 20% to 90%, 20% to 80%, 20% to 70%, 20% to 60%, 20% to 50%, 20% to 40%, 20% to 30%, 30% to 95%, 30% to 90%, 30% to 80%, 30% to 70%, 30% to 60%, 30% to 50%, 30% to 40%, 40% to 95%, 40% to 90%, 40% to 80%, 40% to 70%, 40% to 60%, 40% to 50%, 50% to 95%, 50% to 90%, 50% to 80%, 50% to 70%, 50% to 60%, 60% to 95%, 60% to 90%, 60% to 80%, 60% to 70%, 70% to 95%, 70% to 90%, 70% to 80%, 80% to 95%, 80% to 90%, 90% to 95%, or greater than 95% dendritic cells. In one embodiment, the mixture comprising monocytes and dendritic cells comprises from 10% to 30%, 35% to 55%, 60% to 80%, or 85% to 95% dendritic cells. In one embodiment, the mixture comprising monocytes and dendritic cells comprises at least about 10%, about 15%, about 20%, about 25%, about 30%, about 35%, about 40%, about 45% about 50%, about 55%, about 60%, about 65%, about 70%, about 75%, about 80%, about 85%, about 90%, about 95%, or greater than about 95% dendritic cells.

In one embodiment, the subject is receiving another anti-cancer therapy. In one embodiment, the other anti-cancer therapy comprises treatment with one or more immune checkpoint inhibitors. In one embodiment, the other anti-cancer therapy is an anti-CTLA4 therapy, anti-PD1 therapy, anti-PDL1 therapy, anti-LAG-3 therapy, tumor-treating fields (TTFs), cell-based therapy, a tyrosine kinase inhibitor, a VEGF inhibitor, or any combination thereof. In one embodiment, the other anti-cancer therapy comprises treatment with imatinib, sunitinib, regorafenib, pazopanib, nilotinib, avapritinib, ripretinib, sorafenib, pimitespib, ipilimumab, tremelimumab, nivolumab, pembrolizumab, cemiplimab, atelizumab, avelumab, durvalumab, relatlimab, or any combination thereof.

In one embodiment, the subject does not respond to the other anti-cancer therapy in the absence of administration of the composition comprising antigen presenting cells.

In another aspect, provided herein is a pharmaceutical composition suitable for intratumoral injection, wherein the pharmaceutical composition comprises antigen presenting cells obtained from one or more swine.

### 4. BRIEF DESCRIPTION OF THE DRAWINGS

FIG. 1: FIG. 1 shows a general leukapheresis procedure to generate a leukopak containing antigen presenting cells from porcine blood.

FIG. 2 shows a comparison of tumor size between the treatment group (administration of a composition comprising human PBMCs) and the control in a murine melanoma model at Day 12.

FIG. 3 shows a comparison of tumor size between the treatment group (administration of a composition comprising human PBMCs) and the control in a murine bladder cancer model at Day 7.

FIGS. 4A-4C: FIG. 4A shows human CD4 T cells count at Day 7 in an in vitro study evaluating (a) porcine PBMCs plated with human PBMCs at varying ratios of human to porcine cells (h:p), (b) human PBMCs alone, and (c) allogeneic human PBMCs. FIG. 4B shows human CD8 T cells count at Day 7 in an in vitro study evaluating (a) porcine PBMCs plated with human PBMCs at varying ratios of human to porcine cells (h:p), (b) human PBMCs alone, and (c) allogeneic human PBMCs. FIG. 4C shows human NK cells count at Day 7 in an in vitro study evaluating (a) porcine PBMCs plated with human PBMCs at varying ratios of human to porcine cells (h:p), (b) human PBMCs alone, and (c) allogeneic human PBMCs.

FIGS. 5A-5G: FIG. 5A shows human CD4 T cells count at Day 7 in an in vitro study evaluating (a) porcine monocytes plated with human PBMCs at varying ratios of human to porcine cells (h:p), (b) human PBMCs alone, and (c) allogeneic human PBMCs. FIG. 5B shows human CD8 T cells count at Day 7 in an in vitro study evaluating (a) porcine monocytes plated with human PBMCs at varying ratios of human to porcine cells (h:p), (b) human PBMCs alone, and (c) allogeneic human PBMCs. FIG. 5C shows proliferated human CD4 T cells count at Day 7 in an in vitro study evaluating (a) porcine monocytes plated with human PBMCs at varying ratios of human to porcine cells (h:p), (b) human PBMCs alone, and (c) allogeneic human PBMCs. FIG. 5D shows proliferated human CD8 T cells count at Day 7 in an in vitro study evaluating (a) porcine monocytes plated with human PBMCs at varying ratios of human to porcine cells (h:p), (b) human PBMCs alone, and (c) allogeneic human PBMCs. FIG. 5E shows HLA-DR on human CD4 T cells, in terms of mean fluorescence intensity (MFI), evaluated at Day 7 in an in vitro study evaluating (a) porcine monocytes plated with human PBMCs at varying ratios of human to porcine cells (h:p), (b) human PBMCs alone, and (c) allogeneic human PBMCs. FIG. 5F shows HLA-DR on human CD8 T cells, in terms of mean fluorescence intensity (MFI), evaluated at Day 7 in an in vitro study evaluating (a) porcine monocytes plated with human PBMCs at varying ratios of human to porcine cells (h:p), (b) human PBMCs alone, and (c) allogeneic human PBMCs. FIG. 5G shows human NK cells count at Day 7 in an in vitro study evaluating (a) porcine monocytes plated with human PBMCs at varying ratios of human to porcine cells (h:p), (b) human PBMCs alone, and (c) allogeneic human PBMCs.

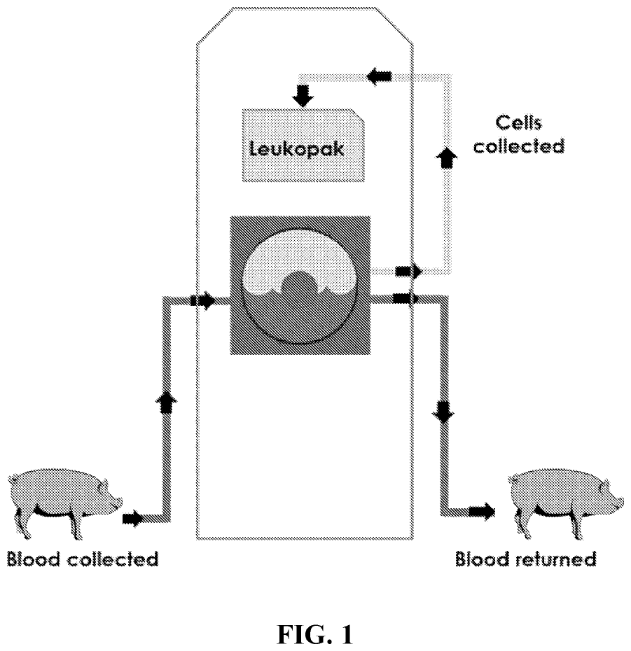

### 5. DETAILED DESCRIPTION OF THE DISCLOSURE

Provided herein are methods for treating a tumor in a subject via administering a composition comprising antigen presenting cells obtained from a species that is different than the subject, i.e., xenogeneic antigen presenting cells. The source of such xenogeneic antigen presenting cells, which could be one or more swine, is described in Section 5.1. Also described in Section 5.1 are characteristics of the swine, and compositions comprising antigen presenting cells, including swine-derived antigen presenting cells. The methods of isolating antigen presenting cells (e.g., from one or more swine) are described in Section 5.2. The methods of treating tumors (e.g., a cancerous solid tumor) comprising administering the antigen presenting cells described herein (e.g., via intratumoral injection), optionally in combination with other anti-cancer therapies, is described in Section 5.3. Furthermore, pharmaceutical compositions comprising the antigen presenting cells disclosed herein are described in Section 5.4.

### 5.1 Xenogeneic Compositions

Provided herein are compositions for treating a tumor in a human subject, wherein the compositions comprise antigen presenting cells derived from a species that is different than the subject. In certain embodiments, provided herein are compositions comprising antigen presenting cells xenogeneic to the human subject to be treated. Such compositions are referred to herein as xenogeneic compositions. The xenogeneic compositions of the present invention may be administered (e.g., by intratumoral injection) either alone or upon formulating into a pharmaceutical composition as described in Section 5.4

The antigen presenting cells in the xenogeneic composition may be any cell that displays antigen bound by major histocompatibility complex (MHC) proteins on its surface. In one embodiment, the MHC proteins are MHC Class II molecules. The antigen presenting cells in the xenogeneic composition may include, but are not limited to, dendritic cells, macrophages, B cells, monocytes, mononuclear phagocytes, endothelial cells, thymic epithelial cells, granulocytes, or any combination thereof.

The xenogeneic compositions of the present invention may comprise antigen presenting cells from any nonhuman species, including, but not limited to, nonhuman primates, domestic animals, and rodents. The nonhuman species may be genetically modified. Domestic animals include pigs, rabbits, dogs, cats, horses, sheep, goats, and cows. In one embodiment, the nonhuman species is a pig or porcine or swine. As provided herein, the terms pig, porcine, or swine may be used interchangeably.

The antigen presenting cells in the xenogeneic compositions of the present invention may be obtained by the methods described in Section 5.2. In certain embodiments, the xenogeneic composition comprises antigen presenting cells derived from one or more pigs. The one or more pigs used for generating the antigen presenting cells could be genetically modified swine, for example, to enhance safety and/or efficacy of the antigen presenting cells. In some embodiments, the swine is an alpha-1,3 galactosyltransferase-deficient swine. In certain embodiments, the antigen presenting cells are derived from one or more recombinant swine. In certain embodiments, the antigen presenting cells are derived from recombinant swine of different genotypes. In certain embodiments, the antigen presenting cells are derived from one or more miniature swine. The one or more miniature swine used for generating the antigen presenting cells could be genetically modified miniature swine, for example, to enhance safety and/or efficacy of the antigen presenting cells. In some embodiments, the miniature swine is an alpha-1,3 galactosyltransferase-deficient miniature swine (see, e.g., U.S. Pat. Nos. 6,153,428, 6,413,769, and 7,547,816). In certain embodiments, the antigen presenting cells are derived from one or more recombinant miniature swine. In certain embodiments, the antigen presenting cells are derived from recombinant miniature swine of different genotypes. In some embodiments, the alpha-1,3 galactosyltransferase-deficient swine or miniature swine is a swine leukocyte antigen (SLA) inbred swine. In certain embodiments, the one or more swine or miniature swine described herein may contain knockout genes or transgenes in the genes of porcine antigen presenting cells.

The donors for the antigen presenting cells used in the xenogeneic compositions described herein can be tested for infectious diseases using methods generally known in the art. In certain embodiments, the donors for the antigen presenting cells used in the xenogeneic compositions described herein tested negative for syphilis, HIV, hepatitis A, hepatitis B, and/or hepatitis C. In certain embodiments, the xenogeneic compositions comprising antigen presenting cells are substantially free of pathogens.

In certain embodiments, the xenogeneic composition comprises antigen presenting cells derived from peripheral blood. In certain embodiments, the xenogeneic composition comprises antigen presenting cells derived from porcine peripheral blood. In certain embodiments, the xenogeneic composition comprises antigen presenting cells derived from bone marrow. In certain embodiments, the xenogeneic composition comprises antigen presenting cells derived from porcine bone marrow.

In certain embodiments, the xenogeneic composition comprises peripheral blood mononuclear cells (PBMCs). In certain embodiments, the xenogeneic composition comprises monocytes. In certain embodiments, the xenogeneic composition comprises porcine PBMCs. In certain embodiments, the xenogeneic composition comprises porcine monocytes. In certain embodiments, the xenogeneic composition comprises monocytes derived from porcine peripheral blood. In certain embodiments, the xenogeneic composition comprises monocytes derived from porcine bone marrow.

In certain embodiments, the xenogeneic composition comprises at least about 50%, about 55%, about 60%, about 65%, about 70%, about 75%, about 80%, about 85%, about 90%, about 95%, or greater than about 95% PBMCs. The PBMCS may be mature, immature, or a combination of mature and immature PBMCs.

In certain embodiments, the xenogeneic composition comprises from 50% to 95%, 50% to 90%, 50% to 80%, 50% to 70%, 50% to 60%, 60% to 95%, 60% to 90%, 60% to 80%, 60% to 70%, 70% to 95%, 70% to 90%, 70% to 80%, 80% to 95%, 80% to 90%, 90% to 95%, or greater than 95% PBMCs.

In certain embodiments, the PBMCs present in the xenogeneic composition are at least from 10% to 95%, 10% to 90%, 10% to 80%, 10% to 70%, 10% to 60%, 10% to 50%, 10% to 40%, 10% to 30%, 10% to 20%, 20% to 95%, 20% to 90%, 20% to 80%, 20% to 70%, 20% to 60%, 20% to 50%, 20% to 40%, 20% to 30%, 30% to 95%, 30% to 90%, 30% to 80%, 30% to 70%, 30% to 60%, 30% to 50%, 30% to 40%, 40% to 95%, 40% to 90%, 40% to 80%, 40% to 70%, 40% to 60%, 40% to 50%, 50% to 95%, 50% to 90%, 50% to 80%, 50% to 70%, 50% to 60%, 60% to 95%, 60% to 90%, 60% to 80%, 60% to 70%, 70% to 95%, 70% to 90%, 70% to 80%, 80% to 95%, 80% to 90%, 90% to 95%, or greater than 95% mature PBMCs.

In certain embodiments, the PBMCs present in the xenogeneic composition are at least from 10% to 30%, 35% to 55%, 60% to 80%, or 85% to 95% mature PBMCs.

In certain embodiments, the PBMCs present in the xenogeneic composition are at least about 10%, about 15%, about 20%, about 25%, about 30%, about 35%, about 40%, about 45% about 50%, about 55%, about 60%, about 65%, about 70%, about 75%, about 80%, about 85%, about 90%, or about 95% mature PBMCs. In certain embodiments, the PBMCs present in the xenogeneic composition are greater than about 95% mature PBMCs. In certain embodiments, the PBMCs present in the xenogeneic composition are about 100% mature PBMCs.

In certain embodiments, the xenogeneic composition comprises at least about 50%, about 55%, about 60%, about 65%, about 70%, about 75%, about 80%, about 85%, about 90%, about 95%, or greater than about 95% monocytes. The monocytes may be mature, immature, or a combination of mature and immature monocytes.

In certain embodiments, the xenogeneic composition comprises from 50% to 95%, 50% to 90%, 50% to 80%, 50% to 70%, 50% to 60%, 60% to 95%, 60% to 90%, 60% to 80%, 60% to 70%, 70% to 95%, 70% to 90%, 70% to 80%, 80% to 95%, 80% to 90%, 90% to 95%, or greater than 95% monocytes.

In certain embodiments, the monocytes present in the xenogeneic composition are at least from 10% to 95%, 10% to 90%, 10% to 80%, 10% to 70%, 10% to 60%, 10% to 50%, 10% to 40%, 10% to 30%, 10% to 20%, 20% to 95%, 20% to 90%, 20% to 80%, 20% to 70%, 20% to 60%, 20% to 50%, 20% to 40%, 20% to 30%, 30% to 95%, 30% to 90%, 30% to 80%, 30% to 70%, 30% to 60%, 30% to 50%, 30% to 40%, 40% to 95%, 40% to 90%, 40% to 80%, 40% to 70%, 40% to 60%, 40% to 50%, 50% to 95%, 50% to 90%, 50% to 80%, 50% to 70%, 50% to 60%, 60% to 95%, 60% to 90%, 60% to 80%, 60% to 70%, 70% to 95%, 70% to 90%, 70% to 80%, 80% to 95%, 80% to 90%, 90% to 95%, or greater than 95% mature monocytes.

In certain embodiments, the monocytes present in the xenogeneic composition are at least from 10% to 30%, 35% to 55%, 60% to 80%, or 85% to 95% mature monocytes.

In certain embodiments, the monocytes present in the xenogeneic composition are at least about 10%, about 15%, about 20%, about 25%, about 30%, about 35%, about 40%, about 45% about 50%, about 55%, about 60%, about 65%, about 70%, about 75%, about 80%, about 85%, about 90%, or about 95% mature monocytes. In certain embodiments, the monocytes present in the xenogeneic composition are greater than about 95% mature monocytes. In certain embodiments, the monocytes present in the xenogeneic composition are about 100% mature monocytes.

In certain embodiments, the xenogeneic composition comprises a mixture of monocytes and dendritic cells. In certain embodiments, the xenogeneic composition comprises a mixture of monocytes and dendritic cells derived from one or more pigs. The composition may further comprise macrophages, granulocytes, B cells, T cells, NK cells, or any combination thereof. The macrophages, granulocytes, B cells, T cells, NK cells, or any combination thereof may be derived from one or more pigs, or other non-human species.

In certain embodiments, the xenogeneic composition comprises a mixture of at least about 50%, about 55%, about 60%, about 65%, about 70%, about 75%, about 80%, about 85%, about 90%, about 95%, or greater than about 95% monocytes and dendritic cells. The monocytes and/or dendritic cells in such mixtures may be mature, immature, or a combination of mature and immature monocytes and/or dendritic cells.

In certain embodiments, the xenogeneic composition comprises a mixture of from 50% to 95%, 50% to 90%, 50% to 80%, 50% to 70%, 50% to 60%, 60% to 95%, 60% to 90%, 60% to 80%, 60% to 70%, 70% to 95%, 70% to 90%, 70% to 80%, 80% to 95%, 80% to 90%, 90% to 95%, or greater than 95% monocytes and dendritic cells.

The mixture of monocytes and dendritic cells may comprise from 10% to 95%, 10% to 90%, 10% to 80%, 10% to 70%, 10% to 60%, 10% to 50%, 10% to 40%, 10% to 30%, 10% to 20%, 20% to 95%, 20% to 90%, 20% to 80%, 20% to 70%, 20% to 60%, 20% to 50%, 20% to 40%, 20% to 30%, 30% to 95%, 30% to 90%, 30% to 80%, 30% to 70%, 30% to 60%, 30% to 50%, 30% to 40%, 40% to 95%, 40% to 90%, 40% to 80%, 40% to 70%, 40% to 60%, 40% to 50%, 50% to 95%, 50% to 90%, 50% to 80%, 50% to 70%, 50% to 60%, 60% to 95%, 60% to 90%, 60% to 80%, 60% to 70%, 70% to 95%, 70% to 90%, 70% to 80%, 80% to 95%, 80% to 90%, 90% to 95%, or greater than 95% monocytes.

In certain embodiments, the mixture of monocytes and dendritic cells comprises from 10% to 30%, 35% to 55%, 60% to 80%, or 85% to 95% monocytes.

In certain embodiments, the mixture of monocytes and dendritic cells comprises at least about 10%, about 15%, about 20%, about 25%, about 30%, about 35%, about 40%, about 45% about 50%, about 55%, about 60%, about 65%, about 70%, about 75%, about 80%, about 85%, about 90%, about 95%, or greater than about 95% monocytes.

In certain embodiments, the monocytes present in the mixture of monocytes and dendritic cells are at least from 10% to 95%, 10% to 90%, 10% to 80%, 10% to 70%, 10% to 60%, 10% to 50%, 10% to 40%, 10% to 30%, 10% to 20%, 20% to 95%, 20% to 90%, 20% to 80%, 20% to 70%, 20% to 60%, 20% to 50%, 20% to 40%, 20% to 30%, 30% to 95%, 30% to 90%, 30% to 80%, 30% to 70%, 30% to 60%, 30% to 50%, 30% to 40%, 40% to 95%, 40% to 90%, 40% to 80%, 40% to 70%, 40% to 60%, 40% to 50%, 50% to 95%, 50% to 90%, 50% to 80%, 50% to 70%, 50% to 60%, 60% to 95%, 60% to 90%, 60% to 80%, 60% to 70%, 70% to 95%, 70% to 90%, 70% to 80%, 80% to 95%, 80% to 90%, 90% to 95%, or greater than 95% mature monocytes.

In certain embodiments, the monocytes present in the mixture of monocytes and dendritic cells are at least from 10% to 30%, 35% to 55%, 60% to 80%, or 85% to 95% mature monocytes.

In certain embodiments, the monocytes present in the mixture of monocytes and dendritic cells are at least about 10%, about 15%, about 20%, about 25%, about 30%, about 35%, about 40%, about 45% about 50%, about 55%, about 60%, about 65%, about 70%, about 75%, about 80%, about 85%, about 90%, or about 95% mature monocytes. In certain embodiments, the monocytes present in the mixture of monocytes and dendritic cells are greater than about 95% mature monocytes. In certain embodiments, the monocytes present in the mixture of monocytes and dendritic cells are about 100% mature monocytes.

The mixture of monocytes and dendritic cells may comprise from 10% to 95%, 10% to 90%, 10% to 80%, 10% to 70%, 10% to 60%, 10% to 50%, 10% to 40%, 10% to 30%, 10% to 20%, 20% to 95%, 20% to 90%, 20% to 80%, 20% to 70%, 20% to 60%, 20% to 50%, 20% to 40%, 20% to 30%, 30% to 95%, 30% to 90%, 30% to 80%, 30% to 70%, 30% to 60%, 30% to 50%, 30% to 40%, 40% to 95%, 40% to 90%, 40% to 80%, 40% to 70%, 40% to 60%, 40% to 50%, 50% to 95%, 50% to 90%, 50% to 80%, 50% to 70%, 50% to 60%, 60% to 95%, 60% to 90%, 60% to 80%, 60% to 70%, 70% to 95%, 70% to 90%, 70% to 80%, 80% to 95%, 80% to 90%, 90% to 95%, or greater than 95% dendritic cells.

In certain embodiments, the mixture of monocytes and dendritic cells comprises from 10% to 30%, 35% to 55%, 60% to 80%, or 85% to 95% dendritic cells.

In certain embodiments, the mixture of monocytes and dendritic cells comprises at least about 10%, about 15%, about 20%, about 25%, about 30%, about 35%, about 40%, about 45% about 50%, about 55%, about 60%, about 65%, about 70%, about 75%, about 80%, about 85%, about 90%, or about 95% dendritic cells. In certain embodiments, the mixture of monocytes and dendritic cells comprises greater than about 95% dendritic cells.

In certain embodiments, the dendritic cells present in the mixture of monocytes and dendritic cells are at least from 10% to 95%, 10% to 90%, 10% to 80%, 10% to 70%, 10% to 60%, 10% to 50%, 10% to 40%, 10% to 30%, 10% to 20%, 20% to 95%, 20% to 90%, 20% to 80%, 20% to 70%, 20% to 60%, 20% to 50%, 20% to 40%, 20% to 30%, 30% to 95%, 30% to 90%, 30% to 80%, 30% to 70%, 30% to 60%, 30% to 50%, 30% to 40%, 40% to 95%, 40% to 90%, 40% to 80%, 40% to 70%, 40% to 60%, 40% to 50%, 50% to 95%, 50% to 90%, 50% to 80%, 50% to 70%, 50% to 60%, 60% to 95%, 60% to 90%, 60% to 80%, 60% to 70%, 70% to 95%, 70% to 90%, 70% to 80%, 80% to 95%, 80% to 90%, 90% to 95%, or greater than 95% mature dendritic cells.

In certain embodiments, the dendritic cells present in the mixture of monocytes and dendritic cells are at least from 10% to 30%, 35% to 55%, 60% to 80%, or 85% to 95% mature dendritic cells.

In certain embodiments, the dendritic cells present in the mixture of monocytes and dendritic cells are at least about 10%, about 15%, about 20%, about 25%, about 30%, about 35%, about 40%, about 45% about 50%, about 55%, about 60%, about 65%, about 70%, about 75%, about 80%, about 85%, about 90%, or about 95% mature dendritic cells. In certain embodiments, the dendritic cells present in the mixture of monocytes and dendritic cells are greater than about 95% mature dendritic cells. In certain embodiments, the dendritic cells present in the mixture of monocytes and dendritic cells are about 100% mature dendritic cells.

In certain embodiments, the antigen presenting cells in the xenogeneic composition are not activated or stimulated. In certain embodiments, the antigen presenting cells in the xenogeneic composition are activated or stimulated. In certain embodiments, the antigen presenting cells are stimulated with one or more of pro-inflammatory factors (e.g., granulocyte macrophage colony-stimulating factor (GM-CSF), interleukin 4 (IL-4), etc.). For example, the porcine monocytes can be cultured into dendritic cells by using GM-CSF and IL-4 for 7 days.

In certain embodiments, the PBMCs in the xenogeneic composition are not activated or stimulated. In certain embodiments, the PBMCs in the xenogeneic composition are activated or stimulated. In certain embodiments, the PBMCs are stimulated with one or more of pro-inflammatory factors.

In certain embodiments, the monocytes and/or dendritic cells in the xenogeneic composition are not activated or stimulated. In certain embodiments, the monocytes and/or dendritic cells in the xenogeneic composition are activated or stimulated. In certain embodiments, the monocytes and/or dendritic cells are stimulated with one or more of pro-inflammatory factors.

In certain embodiments, the antigen presenting cells in the xenogeneic composition are activated or stimulated using porcine B cells as stimulators. In certain embodiments, the antigen presenting cells in the xenogeneic composition are activated or stimulated using porcine T cells as stimulators. In certain embodiments, the antigen presenting cells in the xenogeneic composition are activated or stimulated using porcine monocyte-derived macrophages as stimulators.

### 5.2 Methods of Obtaining Antigen Presenting Cells

Provided herein are methods of obtaining the antigen presenting cells in the xenogeneic compositions described in Section 5.1.

The antigen presenting cells may be derived from tissue or blood of any nonhuman species (e.g., one or more pigs). Such antigen presenting cells may be obtained through methods generally known in the art. The antigen presenting cells may be obtained using a leukapheresis procedure. Such leukapheresis procedure may be used to generate a leukopak, containing peripheral blood mononuclear cells. The leukopak may be further fractionated by counterflow elutriation to obtain a composition comprising the desired antigen presenting cells. In some embodiments, the antigen presenting cells are obtained using fluorescence-activated cell sorting (FACS) or magnetic-activated cell sorting (MACS) techniques.

### 5.3 Methods of Treatment

Provided herein are methods for treating a tumor in a subject in need thereof, comprising administering to the subject (e.g., by intratumoral injection) a xenogeneic composition described in Section 5.1. In certain embodiments, the administering is done via one or more intratumoral injections as described in Section 5.3.1.

In certain embodiments, provided herein are methods for treating a tumor in a subject in need thereof, comprising administering (e.g., by intratumoral injection) a xenogeneic composition in combination with a composition comprising human antigen presenting cells. The antigen presenting cells in the xenogeneic composition and the composition comprising human antigen presenting cells may or may not be activated or stimulated.

The methods of treatment provided herein can be used for treating a wide variety of tumors. In one embodiment, the tumor is a solid tumor. In one embodiment, the tumor is a cancerous tumor. In a specific embodiment, the tumor is a solid cancerous tumor. In certain embodiments, the tumor is resistant to treatment to chemotherapy and/or treatment with another immunotherapy. In certain embodiments, the xenogeneic composition is administered (e.g., by intratumoral injection) as a frontline therapy for treating one or more tumors. In certain other embodiments, the xenogeneic composition is administered to subjects (e.g., by intratumoral injection) who are receiving or may have previously received another anticancer therapy. In one embodiment, the antigen presenting cells in the xenogeneic composition are as disparate as possible from the cells in the tumor tissue. Thus, in one embodiment, the antigen presenting cells in the xenogeneic composition are distinctly different from the cells in the tumor tissue. For example, in one embodiment, a method of treating human liver cancer comprises administration of a porcine xenogeneic composition that does not comprise porcine liver tissue.

Examples of tumors that can be treated with the methods of the present invention include, but are not limited to, sarcomas, carcinomas, lymphomas, breast tumors, prostate tumors, head and neck tumors, glioblastomas, bladder tumors, pancreatic tumors, liver tumors, ovarian tumors, colorectal tumors, pulmonary tumors, cutaneous tumors, lymphoid tumors, gastrointestinal tumors, gastrointestinal stromal tumors, cervical tumors, hepatocellular carcinomas, renal cell carcinomas, melanomas, colorectal carcinomas, esophageal carcinomas, brain tumors, kidney tumors, lung tumors (including non-small cell lung cancer), gastric tumors, bile-duct tumors, uterine tumors, and childhood (pediatric) tumors.

In certain embodiments, provided herein are methods for treating a neoplastic disease in a subject in need thereof, the method comprising administering (e.g., by intratumoral injection) a xenogeneic composition described in Section 5.1.

The xenogeneic compositions as described herein, may be administered to a subject by any route of administration, including, but not limited to, intratumoral, intravenous, intradermal, subcutaneous, intramuscular, or intranodal administration. In one embodiment, the administering is via parenteral administration. In another embodiment, the administering is via intratumoral administration. In such intratumoral administration, the xenogeneic composition is injected directly into a tumor. In yet another embodiment, the administering is via intracerebral administration.

### 5.3.1 Intratumoral Administration

In certain embodiments, the xenogeneic composition as described herein is administered to a subject by one or more intratumoral injections directly into a tumor. Such direct delivery of the therapeutic composition into target tumor lesions offers several advantages over systemic delivery, including increased local concentrations and potentially diminished systemic toxicities.

In one embodiment, the injection is into a single tumor. In another embodiment, the injection is into different sections of the same tumor. In another embodiment, the injection is into multiple tumors. The ability to inject multiple sites may lead to a more robust adaptive immune response (e.g., in patients with polyclonal metastases). In some embodiments, the injection is into a primary tumor. In other embodiments, the injection is into one or more metastatic tumors.

In one embodiment, the intratumoral injection is an image-guided intratumoral injection. The intratumoral injection may be performed under imaging techniques, including, but not limited to, ultrasound, fluoroscopy, and CT scan.

The injection technique, including the design and placement of the injection needle used and the rate of delivery of the therapeutic composition, used for carrying out the intratumoral injection may have an influence on the therapeutic effect (e.g., immune response). In certain embodiments, the intratumoral injection is carried out by placing the needle (e.g., using image guidance) into the target tumor, ensuring that the therapeutic composition is distributed throughout the target tumor without leakage into the surrounding tissue (i.e., without off-target leakage). In one embodiment, the intratumoral injection is carried out by placing the tip of the needle (e.g., using image guidance) within the center of the target tumor.

The intratumoral injection may be performed using a needle having an appropriate needle gauge. The appropriate needle gauge depends on a variety of factors, including, but not limited to, the type of target tumor and its location and size. In certain embodiments, the intratumoral injection is performed using an 18-gauge, 19-gauge, 20-gauge, 21-gauge, 22-gauge, 23-gauge, 24-gauge, 25-gauge, 26-gauge, 27-gauge, 28-gauge, 29-gauge, or 30-gauge needle. In one embodiment, the intratumoral injection is performed using a 20-gauge needle. In one embodiment, the intratumoral injection is performed using a 21-gauge needle. In one embodiment, the intratumoral injection is performed using a 22-gauge needle.

In certain embodiments, the intratumoral injection is performed using an end hole needle (EHN) having an appropriate needle gauge. In one embodiment, the intratumoral injection is performed using a 20-gauge, 21-gauge, or 22-gauge end hole needle (EHN) (e.g., commercialized by Becton Dickinson).

In certain embodiments, the intratumoral injection is performed using a multiside hole needle (MSHN) having an appropriate needle gauge. Placement of such may be performed under image guidance, ensuring that all of the side holes are positioned within the tumor. In one embodiment, the intratumoral injection is performed whereby there is no off-target leakage of the therapeutic composition. In one embodiment, the intratumoral injection is performed using a 20-gauge, 21-gauge, or 22-gauge multiside hole needle (MSHN). In one embodiment, the intratumoral injection is performed using a 21-gauge multiside hole needle (MSHN) with no end hole (e.g., ProFusion™ Therapeutic Infusion Needle commercialized by Cook Regentec).

### 5.3.2 Dosages and Dosing Frequency

The xenogeneic compositions of the present invention may be administered (e.g., by intratumoral injection) in a manner appropriate to the disease to be treated. The dosage amounts and frequency of administration will be determined by such factors as the route of administration, the condition of the patient, and the type and severity of the patient's disease, although appropriate dosages may be determined based on clinical trials.

In certain embodiments, the xenogeneic compositions comprising antigen presenting cells as described herein may be administered (e.g., by intratumoral injection) in single or multiple doses. In one embodiment, the composition is administered as a priming dose. In another embodiment, the composition is administered under a booster regimen (i.e., repeated treatment). In yet another embodiment, the composition is administered under a booster regimen (i.e., repeated treatment), but in a different tumor site.

In certain embodiments, the multiple doses may be administered on the same day at essentially the same time, immediately following the previous dose (i.e., back-to-back administration). Alternatively, the multiple doses may be administered on the same day but not at the same time (i.e., not back-to-back administration). In such instances, the multiple doses may be 2 doses, 3 doses, 4 doses, 5 doses, 6 doses, 7 doses, 8 doses, 9 doses, or 10 doses.

In certain embodiments, the administration of the xenogeneic composition as described herein may be repeated and separated by at least 1 day, 2 days, 3 days, 4 days, 5 days, 7 days, 10 days, 15 days, 30 days, 45 days, 2 months, 75 days, 3 months, or at least 6 months. In some embodiments, dosing can be discontinued for a predetermined number of days.

In certain embodiments, the composition as described herein is administered once a week for a predetermined number of weeks. In one embodiment, the scheduled number of weeks is 1 week, 2 weeks, 3 weeks, 4 weeks, 5 weeks, 6 weeks, 7 weeks, 8 weeks, 9 weeks, or 10 weeks. In some embodiments, dosing can be discontinued for at least one week. In certain embodiments, the composition is administered as a single dose followed by a second dose one to six weeks later. In certain embodiments, the composition may be administered at six to twelve months intervals.

When the composition as described herein is administered multiple times, the individual doses may be essentially of the same dosage amount or different dosage amounts. Furthermore, the composition may be administered (e.g., via intratumoral injection) at the same tumor site or different tumor sites.

The xenogeneic compositions of the present invention may be administered in varying dosage amounts, depending on, inter alia, the route of administration, the condition of the patient, and the type and severity of the patient's disease, although appropriate dosages may be determined based on clinical trials.

In certain embodiments, the xenogeneic composition is administered via one or more (e.g., intratumoral) injections of from 1×10⁶ to 10×10⁶, 10×10⁶ to 20×10⁶, 20×10⁶ to 30×10⁶, 30×10⁶ to 40×10⁶, 40×10⁶ to 50×10⁶, 50×10⁶ to 60×10⁶, 60×10⁶ to 70×10⁶, 70×10⁶ to 80×10⁶, 80×10⁶ to 90×10⁶, 90×10⁶ to 10×10⁷, 10×10⁷ to 20×10⁷, 20×10⁷ to 30×10⁷, 30×10⁷ to 40×10⁷, 40×10⁷ to 50×10⁷, 50×10⁷ to 60×10⁷, 60×10⁷ to 70×10⁷, 70×10⁷ to 80×10⁷, 80×10⁷ to 90×10⁷, 90×10⁷ to 10×10⁸, 10×10⁸ to 20×10⁸, 20×10⁸ to 30×10⁸, 30×10⁸ to 40×10⁸, 40×10⁸ to 50×10⁸ antigen presenting cells per dose.

In certain embodiments, the xenogeneic composition is administered via one or more (e.g., intratumoral) injections of about 1×10⁶, about 5×10⁶, about 10×10⁶, about 15×10⁶, about 20×10⁶, about 25×10⁶, about 30×10⁶, about 35×10⁶, about 40×10⁶, about 45×10⁶, about 50×10⁶, about 55×10⁶, about 60×10⁶, about 65×10⁶, about 70×10⁶, about 75×10⁶, about 80×10⁶, about 85×10⁶, about 90×10⁶, about 95×10⁶, about 10×10⁷, about 15×10⁷, about 20×10⁷, about 25×10⁷, about 30×10⁷, about 35×10⁷, about 40×10⁷, about 45×10⁷, about 50×10⁷, about 55×10⁷, about 60×10⁷, about 65×10⁷, about 70×10⁷, about 75×10⁷, about 80×10⁷, about 85×10⁷, about 90×10⁷, about 95×10⁷, about 10×10⁸, about 15×10⁸, about 20×10⁸, about 25×10⁸, about 30×10⁸, about 35×10⁸, about 40×10⁸, about 45×10⁸, or about 50×10⁸ antigen presenting cells per dose. In certain embodiments, the xenogeneic composition is administered via one or more (e.g., intratumoral) injections of about 100,000 and/or about 500,000 antigen presenting cells per dose. In some embodiments, the antigen presenting cells in such compositions are mature antigen presenting cells. In some embodiments, the antigen presenting cells in such compositions are mature antigen presenting cells in combination with immature antigen presenting cells.

A single dose of antigen presenting cells may be administered via one or more (e.g., intratumoral) injections. In certain embodiments, the xenogeneic composition is administered via one or more (e.g., intratumoral) injections of from 1×10⁶ to 10×10⁶, 10×10⁶ to 20×10⁶, 20×10⁶ to 30×10⁶, 30×10⁶ to 40×10⁶, 40×10⁶ to 50×10⁶, 50×10⁶ to 60×10⁶, 60×10⁶ to 70×10⁶, 70×10⁶ to 80×10⁶, 80×10⁶ to 90×10⁶, 90×10⁶ to 10×10⁷, 10×10⁷ to 20×10⁷, 20×10⁷ to 30×10⁷, 30×10⁷ to 40×10⁷, 40×10⁷ to 50×10⁷, 50×10⁷ to 60×10⁷, 60×10⁷ to 70×10⁷, 70×10⁷ to 80×10⁷, 80×10⁷ to 90×10⁷, 90×10⁷ to 10×10⁸, 10×10⁸ to 20×10⁸, 20×10⁸ to 30×10⁸, 30×10⁸ to 40×10⁸, 40×10⁸ to 50×10⁸ antigen presenting cells per injection. In certain embodiments, the xenogeneic composition is administered via one or more (e.g., intratumoral) injections of about 1×10⁶, about 5×10⁶, about 10×10⁶, about 15×10⁶, about 20×10⁶, about 25×10⁶, about 30×10⁶, about 35×10⁶, about 40×10⁶, about 45×10⁶, about 50×10⁶, about 55×10⁶, about 60×10⁶, about 65×10⁶, about 70×10⁶, about 75×10⁶, about 80×10⁶, about 85×10⁶, about 90×10⁶, about 95×10⁶, about 10×10⁷, about 15×10⁷, about 20×10⁷, about 25×10⁷, about 30×10⁷, about 35×10⁷, about 40×10⁷, about 45×10⁷, about 50×10⁷, about 55×10⁷, about 60×10⁷, about 65×10⁷, about 70×10⁷, about 75×10⁷, about 80×10⁷, about 85×10⁷, about 90×10⁷, about 95×10⁷, about 10×10⁸, about 15×10⁸, about 20×10⁸, about 25×10⁸, about 30×10⁸, about 35×10⁸, about 40×10⁸, about 45×10⁸, or about 50×10⁸ antigen presenting cells per injection. In certain embodiments, the xenogeneic composition is administered via one or more (e.g., intratumoral) injections of about 100,000 and/or about 500,000 antigen presenting cells per injections.

### 5.3.3 Clinical Outcomes

The therapeutic effect observed upon administration of the xenogeneic composition of the present invention, individually or in combination with another anticancer therapy, may be assessed by any of the methods commonly used in anticancer therapies. In certain embodiments, the administration of the xenogeneic composition triggers an immune response specific to the targeted tumor.

Furthermore, treatment with the xenogeneic composition described herein, individually or in combination with another anticancer therapy, may shrink not only the targeted tumor but also lead to shrinkage of untreated tumors elsewhere in the body. Such an outcome is known as abscopal effect. Thus, treatment with the xenogeneic composition described herein, individually or in combination with another anticancer therapy, may induce a systemic anti-tumor immune response. In certain embodiments, administration of the xenogeneic composition described herein via a targeted intratumoral injection leads to an abscopal effect. In certain embodiments, administration of the xenogeneic composition described herein via a targeted intratumoral injection in combination with another anticancer therapy (e.g., tumor-treating fields (TTFs) or radiation) leads to an abscopal effect.

In certain embodiments, the therapeutic effect observed upon administration of the xenogeneic composition of the present invention, individually or in combination with another anticancer therapy, is assessed by measuring immunologic response. In certain embodiments, the immunologic response is evaluated by measuring immunologic markers in blood. In certain embodiments, tumor-specific immunological response is evaluated. In certain embodiments, systemic immunological response is evaluated. The immunological response may be evaluated via
cell tracking (which may involve staining PBMCs with specific antibodies, for example, for one HLA class I or one HLA-class II antigen that is selectively expressed on donor vaccine cells);
evaluating dendritic cell-induced alloimmunization (which may involve screening of alloantibodies against, for example, HLA-A, B, C (MHC-class I) and HLA-DR, DQ, DP (MHC-class II) antigens);
monitoring autoimmune events (which may involve screening of autoantibodies against autoantigens, including nuclear antigens (e.g., ANA, SSA, SSB, Sm, RNP, Scl-70, Centromeres, and Jo-1) and liver parenchyma-associated autoantigens (e.g., liver-kidney microsomal antigens and mitochondrial antigens)·
assessing complement activation and/or classical/alternative complement function; and/or
evaluating immune cell occurrence and activation state (e.g., CD3+, CD3+4+ and CD3+8+ T cells, CD19+ B-cells CD3-16+56+ NK-cells, CD3-16+56+69+ NK cells, CD3+16+56+ NKT-cells, CD3+16+56+69+ NKT-cells and CD3+HLA-DR+ T cells).

In certain embodiments, the therapeutic effect observed upon administration of the xenogeneic composition of the present invention, individually or in combination with another anticancer therapy, is assessed by evaluating the size of the tumor/tumors. This may be done after 3 and/or 6 months from administration of the treatment, or as deemed necessary based on tumor progression.

In certain embodiments, the therapeutic effect observed upon administration of the xenogeneic composition of the present invention, individually or in combination with another anticancer therapy, is assessed by evaluating tumor control. In some embodiments, tumor control is evaluated by CT and/or MRI scans, measuring number of tumor specific T cells, measuring AFP (alpha-feto protein) levels in blood, measuring the level of circulating tumor cell, or any combination thereof.

In certain embodiments, the therapeutic effect observed upon administration of the xenogeneic composition of the present invention, individually or in combination with another anticancer therapy, is assessed by evaluating systemic inflammatory response. This may be done by evaluating, for example, systemic release of relevant cytokines, chemokines, and other inflammatory parameters in blood (e.g., IL-1R, IL-2, IL-4, IL-5, IL-6, IL-7, IL-8, IL-10, IL-12p70, IL-13, IL-17A, G-CSF, GM-CSF, IFN-gamma, MCP-1, MIP-1 beta and TNF-alpha).

In certain embodiments, the therapeutic effect is assessed by evaluating long term changes in Eastern Cooperative Oncology Group (ECOG) and/or Karnofsky performance status (KPS) scores. In certain embodiments, the therapeutic effect is assessed by evaluating long term changes in Quality-of-Life scores.

In certain embodiments, the therapeutic effect is assessed in terms of partial response and/or complete response. In certain embodiments, the therapeutic effect is assessed in terms of progression free survival and/or overall survival. In certain embodiments, progression-free survival and/or overall survival is evaluated by measuring blood parameters, for example, by measuring levels of lactate dehydrogenase (LDH) and derived neutrophil-to-lymphocyte ratio (dNLR). Elevated LDH and dNLR may be associated with poorer survival outcomes in patients treated with immunotherapy. In certain embodiments, administration of the xenogeneic composition of the present invention, individually or in combination with another anticancer therapy, results in reduced LDH and dNLR levels relative to baseline.

In certain embodiments, the therapeutic effect is assessed in terms of adverse events, registered as a measure of safety and tolerability, which may include changes in vital signs from baseline (e.g., heart rate, blood pressure, body temperature), changes in laboratory parameters from baseline, etc. In one embodiment, administration of the xenogeneic composition of the present invention, individually or in combination with another anticancer therapy, is not associated with severe adverse events.

### 5.3.4 Combination Treatment

When used in treating cancer, the xenogeneic compositions as described herein may be combined with other therapies. In some embodiments, the xenogeneic composition is administered in combination with therapies decreasing the immunosuppresive tumor environment or activating the immune system.

In certain embodiments, the xenogeneic composition is administered (e.g., by intratumoral injection) in combination with one or more other anticancer therapies. Examples of such other anti-cancer therapies include, but are not limited to, anti-CTLA4 therapy, anti-PD1 therapy, anti-PDL1 therapy, anti-LAG-3 therapy, tumor-treating fields (TTFs), cell-based therapy, a tyrosine kinase inhibitor, a VEGF inhibitor, or any combination thereof. In one embodiment, the other anti-cancer therapy comprises treatment with one or more biologics. In one embodiment, the other anti-cancer therapy comprises treatment with cellular therapy products or gene therapy products. In one embodiment, the other anti-cancer therapy is CAR T-cell based therapy. In one embodiment, the other anti-cancer therapy comprises treatment with T-cell engagers. In one embodiment, the other anti-cancer therapy comprises treatment with one or more immune checkpoint inhibitors. In one embodiment, the other anti-cancer therapy comprises treatment with one or more small-molecule drugs. In one embodiment, the other anti-cancer therapy comprises treatment with one or more protein kinase inhibitors. In one embodiment, the other anti-cancer therapy comprises treatment with one or more tyrosine kinase inhibitors. In one embodiment, the other anti-cancer therapy comprises treatment with one or more alkylating agents, antimetabolites, natural products, or hormones. In one embodiment, the other anti-cancer therapy comprises treatment with gemcitabine or 5-fluorouracil. In one embodiment, the other anti-cancer therapy comprises surgery or radiation therapy. In one embodiment, the other anti-cancer therapy comprises tumor-treating fields.

In certain embodiments, the other anti-cancer therapy comprises treatment with imatinib, sunitinib, regorafenib, pazopanib, nilotinib, avapritinib, ripretinib, sorafenib, pimitespib, ipilimumab, tremelimumab, nivolumab, pembrolizumab, cemiplimab, atelizumab, avelumab, durvalumab, relatlimab, or any combination thereof.

The combination treatments as described herein may provide “synergy” and prove “synergistic,” i.e., the therapeutic effect achieved following administration of two or more individual therapies used together is greater than the sum of the therapeutic effects achieved following administration of the individual therapies separately. Such synergy may be determined by methods commonly known in the art. In certain embodiments, the combination treatment (i.e., administration of the xenogeneic composition of the present invention in combination with another anticancer therapy) provides a synergistic antitumor effect.

Suitable dosages of the individual therapies and/or compositions may be lowered due to the combined action (synergy), so as to increase the therapeutic index or mitigate toxicity or other side-effects or consequences.

### 5.4 Pharmaceutical Compositions

Provided herein are pharmaceutical compositions comprising the xenogeneic compositions described in Section 5.1.

In certain embodiments, provided herein are pharmaceutical compositions comprising the xenogeneic compositions in combination with one or more pharmaceutically or physiologically acceptable carriers, diluents, and/or excipients. Such pharmaceutical compositions can be formulated according to standard procedures in the art. In one embodiment, the pharmaceutical composition is in the form of an aqueous solution.

In certain embodiments, the pharmaceutical composition comprises one or more pharmaceutically acceptable carriers. In some embodiments, the pharmaceutically acceptable carrier is a phosphate buffered saline solution, water, or an emulsion, such as an oil/water or water/oil emulsion. In certain embodiments, the pharmaceutical composition is formulated as an aqueous solution, for example, in physiologically compatible buffers such as Hanks's solution, Ringer's solution, or physiological saline buffer.

In certain embodiments, the pharmaceutical composition comprises pharmaceutically acceptable adjuvants, excipients, stabilizers, preservatives, and/or other components known in the art. In one embodiment, the pharmaceutical composition comprises a wetting agent. In some embodiments, the pharmaceutical composition comprises one or more suspending, stabilizing, and/or dispersing agents. In some embodiments, the pharmaceutical composition comprises one or more emulsifiers. In certain embodiments, the pharmaceutical composition is sterilized. In one embodiment, the pharmaceutical composition comprises one or more preservatives. In another embodiment, the pharmaceutical composition does not contain a preservative.

In certain embodiments, the pharmaceutical compositions provided herein may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and/or preservatives.

In certain embodiments, the pharmaceutical composition comprising the antigen presenting cells as described herein is in the form of a suspension or dispersion. Such a suspension or dispersion may be prepared by means of conventional dispersing and suspending processes, and may contain one or more pharmaceutically acceptable excipients. In one embodiment, the pharmaceutical composition is an isotonic aqueous suspension or dispersion. In one embodiment, the suspension or dispersion comprises one or more viscosity-regulating agents.

In certain embodiments, the pharmaceutical composition comprising the xenogeneic composition as described herein is formulated in the form of a hydrogel. In some embodiments, the hydrogel is a multidomain peptide-based hydrogel. Such multidomain peptide-based hydrogels may be prepared by methods known in the art. In certain embodiments, the antigen presenting cells embedded within a multidomain peptide-based hydrogel exhibit significantly improved delivery and retention within tumors. In certain embodiments, the pharmaceutical composition is a hydrogel-based sustained release composition.

In certain embodiments, the xenogeneic compositions described herein are formulated into a pharmaceutical composition with diluents and/or with other components such as IL-2 or other cytokines or cell populations.

The pharmaceutical compositions described herein may be kept at temperatures around 2-8° C., or may be frozen and then thawed shortly before use. In certain embodiments, the pharmaceutically acceptable carrier may be a medium preserving frozen cells. For example, the xenogeneic compositions comprising antigen presenting cells described herein may be frozen in heat inactivated universal donor plasma comprising dimethyl sulfoxide (DMSO) to allow for storage thereof. Such a medium comprising the antigen presenting cells may be used directly, e.g., injected intratumorally, once thawed. Alternatively, cells frozen in such a medium may be thawed, washed, and re-suspended in an appropriately buffered saline solution or a saline solution comprising human serum albumin before being administered, e.g., injected intratumorally. In one embodiment, the pharmaceutically acceptable carrier is a saline solution comprising human serum albumin. In another embodiment, the pharmaceutically acceptable carrier is a physiological sodium chloride solution comprising 2% human serum albumin.

The pharmaceutical compositions provided herein may be formulated for any route of administration, including, but not limited to, intratumoral, intravenous, intradermal, subcutaneous, intramuscular, or intranodal administration. In one embodiment, the pharmaceutical composition is formulated for parenteral administration. In another embodiment, the pharmaceutical composition is formulated for intratumoral administration. In intratumoral administration, the pharmaceutical composition is injected directly into a tumor. In a specific embodiment, the pharmaceutical composition as described herein is administered by one or more intratumoral injections directly into a tumor. The injection may be to a single tumor or more than one tumor.

The pharmaceutical compositions of the present invention may be administered in a manner appropriate to the disease to be treated. The dosage amounts and frequency of administration will be determined by such factors as the route of administration, the condition of the patient, and the type and severity of the patient's disease, although appropriate dosages may be determined based on clinical trials.

The following examples are offered by way of illustration, and not by way of limitation.

### 6. EXAMPLES

### 6.1 Example 1: Xenogeneic Compositions Comprising Porcine Antigen Presenting Cells

### 6.1.1 Isolation of Peripheral Blood Mononuclear Cells

Porcine blood (~100 ml total volume) collected in EDTA vacutainers (BD Biosciences, San Diego, USA) was diluted 1:1 with Phosphate Buffered Saline (PBS). 30 ml of the diluted blood was carefully layered on 20 ml of Ficoll Paque Plus (Cytiva, Uppsala, Sweden) in a 50 ml Falcon tube. Density gradient centrifugation was performed at 600×g for 20 min with the centrifuge brakes turned off. After centrifugation, Peripheral Blood Mononuclear Cells (PBMC) were collected from the interphase and washed three times with PBS. Each wash consisted of a 5 min centrifugation step at 350×g followed by replacement of supernatant with fresh PBS. After the final wash, PBMC were resuspended in 5 ml of ACK Lysing Buffer (Gibco, Thermo Fisher Scientific Inc., Waltham, USA) and incubated for 5 min. After lysis of red blood cells, PBMC were centrifuged and resuspended in AIM V Medium (Gibco, Thermo Fisher Scientific Inc., Waltham, USA) supplemented with 10% heat-inactivated Fetal Bovine Serum (FBS; Gibco, Thermo Fisher Scientific Inc., Waltham, USA). Human PBMC were similarly purified from anonymous healthy donor buffy coats obtained from the Karolinska University Hospital Blood Bank (Stockholm, Sweden).

### 6.1.2 Isolation of Porcine CD14+ Cells

Porcine CD14+ cells were isolated via positive magnetic bead selection using a cross-reactive anti-human CD14 MicroBeads isolation kit (Miltenyi, Bergisch Gladbach, Germany) according to the manufacturer's instructions. In two separate purifications starting from 1×10⁸ porcine PBMC, the positively selected CD14+ yield amounted to 20% of starting cell count. Cell viability of both PBMC and CD14+ fraction after positive selection was >93% when analyzed using Trypan Blue exclusion (Gibco, Thermo Fisher Scientific Inc., Waltham, USA).

### 6.2 Example 2: Mouse In Vivo Experiment Using Human PBMCs-Murine Melanoma Model

Human peripheral blood mononuclear cells (PBMCs) were isolated from peripheral blood using the Ficoll density gradient centrifugation method.

PBMCs were then labeled with CFSE (carboxyfluorescein succinimidyl ester). CFSE-labeled PBMCs were intratumorally injected at a dose of approximately 1×10⁶ cells into C57BL/6J mice expressing B16 melanoma cell line.

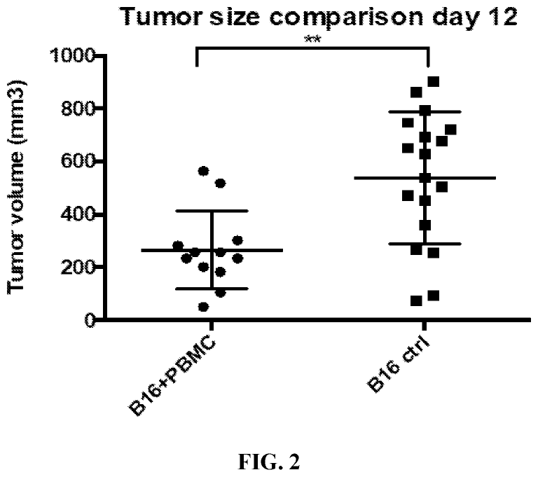

Comparison of the tumor size between the treatment group and the control was conducted by measuring tumor volume at day 12. The results are shown in the table below, and in FIG. 2.

| | Mean tumor size |
|---|---|
| Treatment (n = 12) | 265.8 mm³ |
| Control (n = 18) | 538.2 mm³ |

### 6.3 Example 3: Mouse In Vivo Experiment Using Human PBMCs-Murine Bladder Cancer Model

Human PBMCs were isolated from peripheral blood using the Ficoll density gradient centrifugation method.

PBMCs were then labeled with CFSE (carboxyfluorescein succinimidyl ester). CFSE-labeled PBMCs were intratumorally injected at a dose of approximately 1×10⁶ cells into C57BL/6J mice expressing MB49 carcinogen induced bladder cancer cell line.

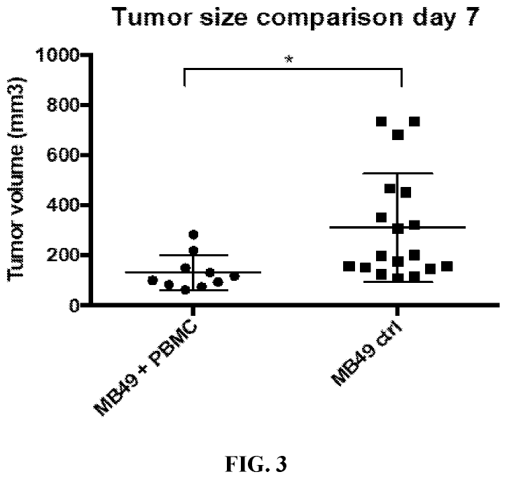

Comparison of the tumor size between the treatment group and the control was conducted by measuring tumor volume at day 7. The results are shown in the table below, and in FIG. 3.

| | Mean tumor size |
|---|---|
| Treatment (n = 10) | 132.0 mm³ |
| Control (n = 18) | 310.6 mm³ |

### 6.4 Example 4: Mixed Lymphocyte Reaction

Human PBMC were stained with Violet Proliferation Dye 450 (VPD450; BD Biosciences, San Diego, USA) following the manufacturer's instructions. In the first xenogeneic Mixed Lymphocyte Reaction (MLR) study, VPD450-stained human PBMC were mixed with unstained porcine PBMC at various ratios. Specifically, starting from a 10:10 human to porcine cell ratio, and proceeding with a fixed number of human cells combined with gradually decreasing numbers of porcine cells to reach a 10:1 human to porcine cell ratio. The 10:10 ratio wells contained a combination of 2×10⁵ human PBMC and 2×10⁵ porcine PBMC, while the 10:1 human to porcine cell ratio wells contained a combination of 2×10⁵ human PBMC and 2×10⁴ porcine PBMC. In the second xenogeneic study, VPD450-stained human PBMC were mixed with unstained porcine CD14+ cells at various ratios, as described above. All samples were resuspended into round-bottom 96-well cell culture plates in 200 μl AIM V Medium supplemented with 10% heat-inactivated FBS and incubated at 37° C. and 5% CO₂ for seven days. On day five, 100 μL fresh culture medium was added to each well. Both xenogeneic MLR studies included two (n=2) PBMC donors separately combined with a single porcine donor. Cells from neither donor/species were inactivated via irradiation/chemical treatment, resulting in a two-way xenogeneic MLR, however, only human cells were labeled with the proliferation dye. Control wells included allogeneic MLR consisting of 2×10⁴ human PBMC combined with 2×10⁴ human PBMC from an unrelated donor. Additional control wells received 2×10⁴ human PBMC from a single donor.

On day seven of the MLR, cells were washed twice in saline solution and incubated with Fc-receptor Binding Inhibitor (Invitrogen; Thermo Fisher Scientific Inc., Waltham, USA) followed by staining anti-CD3 VioGreen (Miltenyi, Bergisch Gladbach, Germany; Clone REA613), anti-CD45RA BV650 (BD Biosciences, San Diego, USA; Clone HI100), anti-CD8 BB515 (BD Biosciences, San Diego, USA; Clone RPA-T8), anti-CCR7 PE (Miltenyi, Bergisch Gladbach, Germany; Clone REA546), anti-HLA-DR PerCP-Cy5.5 (BD Biosciences, San Diego, USA; Clone G46-6), anti-CD4 APC-Vio770 (Miltenyi, Bergisch Gladbach, Germany; Clone REA623), and anti-CD56 APC (Miltenyi, Bergisch Gladbach, Germany; Clone REA196). Samples were stained while resuspended in Stain Buffer (FBS) (BD Biosciences, San Diego, USA), incubated in the dark at 4° C., and, after two washing steps, were acquired using a FACS Celesta flow cytometer (BD Biosciences, San Diego, USA). Post-acquisition data analysis was conducted using FlowJo 10.8.1 software (FlowJo LLC, Ashland, USA).

### 6.5 Example 5: In Vitro Study Comparison: Porcine PBMC Vs Human PBMC

Porcine PBMCs were plated with human PBMCs at varying ratios of human to porcine cells (h:p) as described in the MLR study above. The ratios of human to porcine cells (h:p) were h:p=10:10, 10:9, 10:8, 10:7, 10:6, 10:5, 10:4, 10:3, 10:2, and 10:1. Separately, human PBMCs alone and allogeneic human PBMCs were evaluated as reference samples.

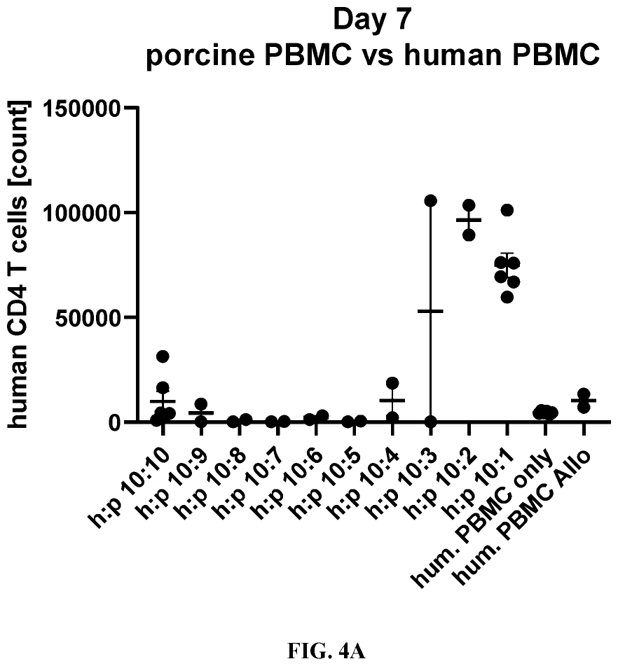

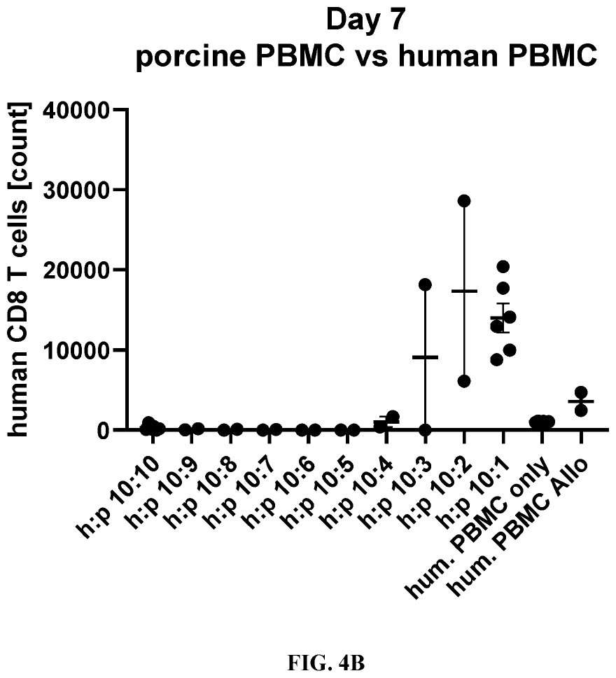

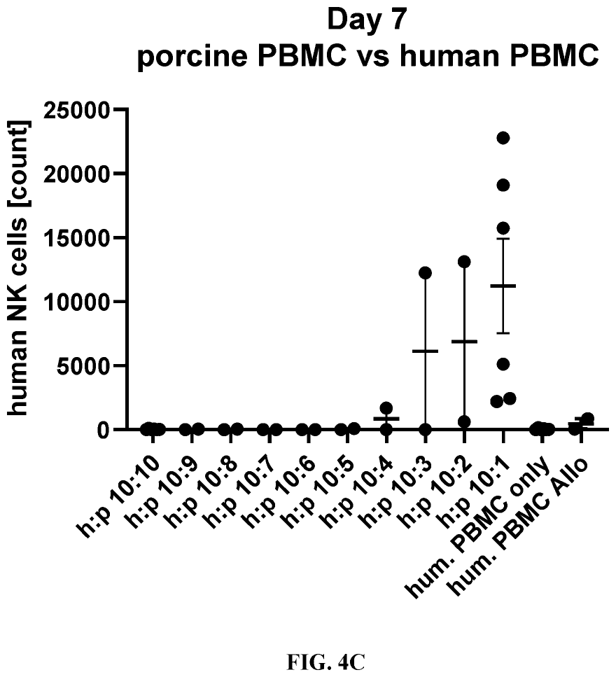

Human CD4 T cells count and human CD8 T cells count were evaluated on Day 7 for all samples, including the reference samples. The results are shown in FIG. 4A and FIG. 4B, respectively.

Human NK cells count was evaluated on Day 7 for all samples, including the reference samples. The results are shown in FIG. 4C.

### 6.6 Example 6: In Vitro Study Comparison: Porcine Monocytes (CD14+ Cells) Vs Human PBMC

Porcine monocytes (containing CD14+ cells) were plated with human PBMCs at varying ratios of human to porcine cells (h:p) as described in the MLR study above. The ratios of human to porcine cells (h:p) were h:p=10:10, 10:9, 10:8, 10:7, 10:6, 10:5, 10:4, 10:3, 10:2, and 10:1. Separately, human PBMCs alone and allogeneic human PBMCs were evaluated as reference samples.

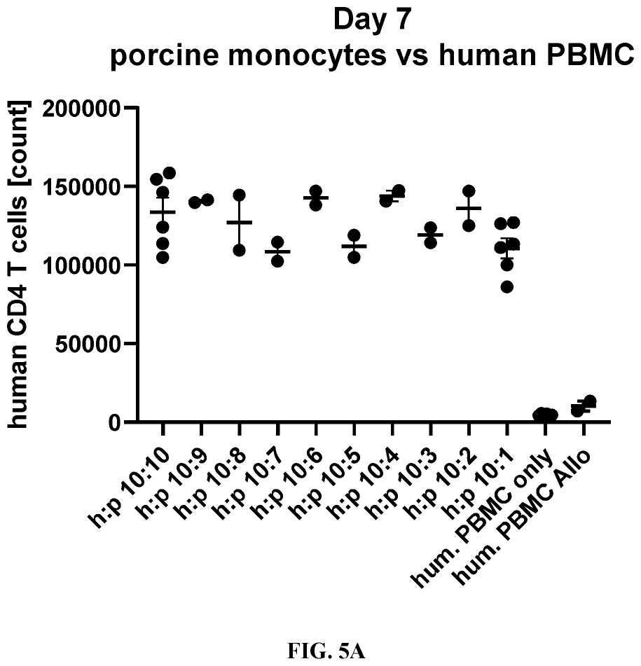

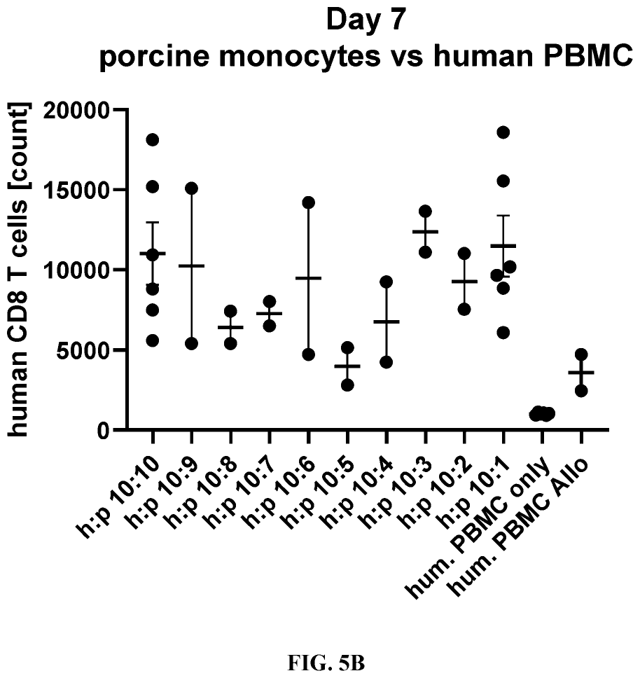

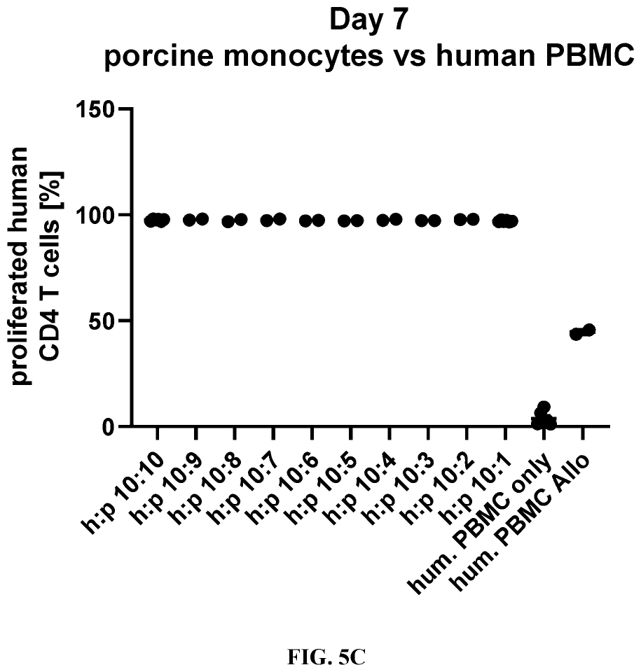

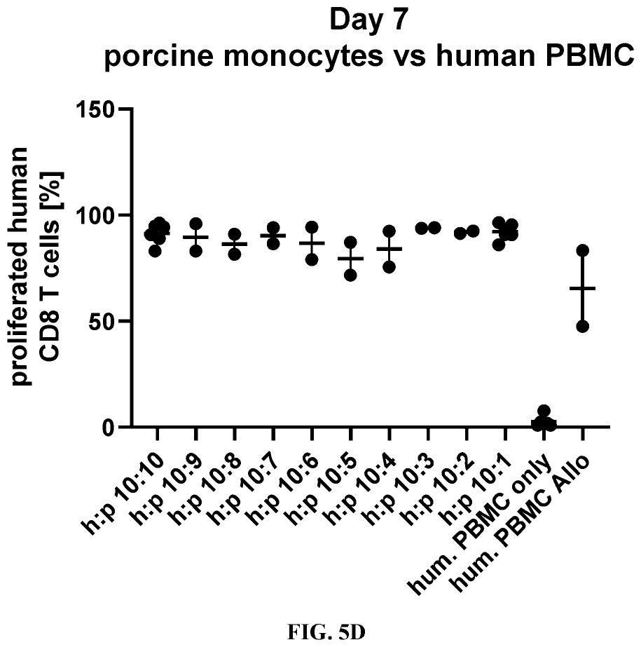

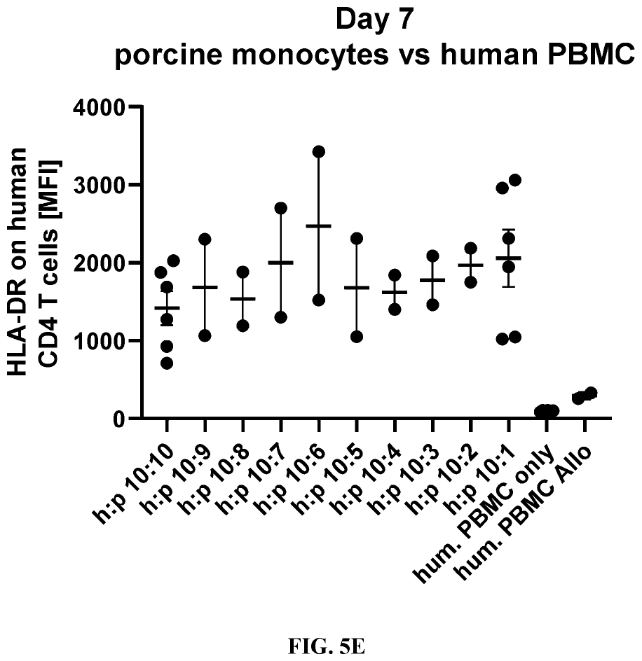

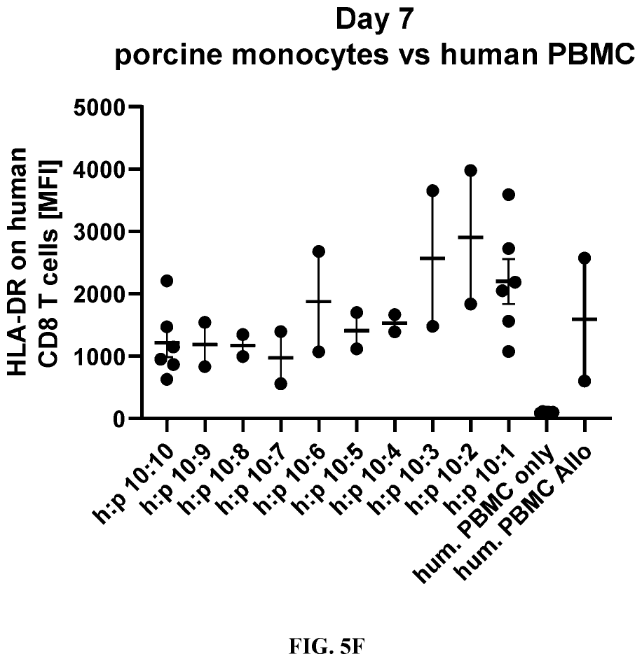

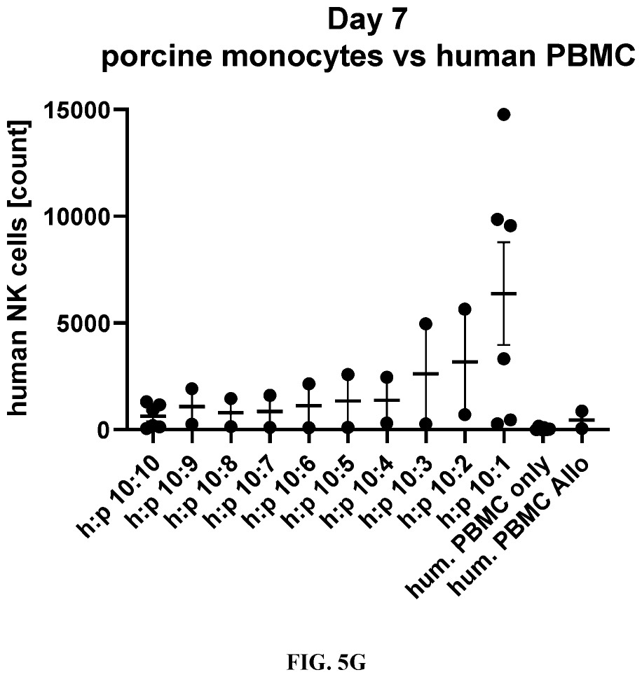

Human CD4 T cells count and human CD8 T cells count were evaluated on Day 7 for all samples, including the reference samples. The results are shown in FIG. 5A and FIG. 5B, respectively.

Proliferated human CD4 T cells count and proliferated human CD8 T cells count were also evaluated on Day 7 for all samples, including the reference samples. The results are shown in FIG. 5C and FIG. 5D, respectively.

HLA-DR on human CD4 T cells and human CD8 T cells, in terms of mean fluorescence intensity (MFI), was evaluated on Day 7 for all samples, including the reference samples. The results are shown in FIG. 5E and FIG. 5F, respectively.

Human NK cells count was evaluated on Day 7 for all samples, including the reference samples. The results are shown in FIG. 5G.

### 6.7 Example 7: Mouse In Vivo Experiment Using Porcine PBMCs

Porcine PBMCs are intratumorally injected at a dose of 1×10⁶ cells into C57BL/6J mice expressing B16 melanoma cell line. Comparison of the tumor size between the treatment group and the control is conducted by measuring tumor volume at day 7 or later.

A similar experiment is conducted in C57BL/6J mice expressing MB49 carcinogen induced bladder cancer cell line.

### 6.8 Example 8: Intratumoral Administration of Porcine PBMCs to Human Patients

This example evaluates the safety and/or efficacy of a composition comprising porcine antigen presenting cells administered intratumorally as single agent or in combination with another anticancer therapy in cancer patients.

Patients are divided in two treatment groups. The first group of patients will receive the treatment composition as monotherapy, whereas the second group of patients will receive the treatment composition in combination with a checkpoint inhibitor or a tyrosine kinase inhibitor. Patients will be treated with the treatment composition at an increasing dose and/or frequency, starting with a dose of 10×10⁶ cells per injection. Patients may or may not have previously been treated with an anticancer therapy. Certain outcome measures are provided below.

Adverse events will be registered as a measure of safety and tolerability, which will include changes in vital signs from baseline (e.g., heart rate, blood pressure, body temperature), changes in laboratory parameters from baseline, etc.

Immunologic response will be evaluated by measuring immunologic markers in blood. The size of the tumor/tumors will be evaluated after 3 and 6 months or as deemed necessary based on tumor progression.

Systemic inflammatory response will be evaluated, including potential systemic release of relevant cytokines, chemokines and other inflammatory parameters in blood (e.g., IL-1R, IL-2, IL-4, IL-5, IL-6, IL-7, IL-8, IL-10, IL-12p70, IL-13, IL-17A, G-CSF, GM-CSF, IFN-gamma, MCP-1, MIP-1 beta and TNF-alpha).

Tumor control will be evaluated, which will include CT/MRI scans, measuring number of tumor specific T cells, measuring AFP (alpha-feto protein) levels in blood, and/or measuring the level of circulating tumor cell.

Tumor-specific immunological responses and systemic immunological response will be evaluated.

Long term changes in Eastern Cooperative Oncology Group (ECOG) and/or Karnofsky performance status (KPS) scores will be evaluated. Further, long term changes in Quality of Life scores will be evaluated.

Therapeutic effect will be assessed in terms of partial response, complete response, progression free survival, and/or overall survival.

### 7. EQUIVALENTS

Although the disclosure is described in detail with reference to specific embodiments thereof, it will be understood that variations that are functionally equivalent are within the scope of this disclosure. Indeed, various modifications of the disclosure in addition to those shown and described herein will become apparent to those skilled in the art from the foregoing description. Such modifications are intended to fall within the scope of the appended claims. Those skilled in the art will recognize, or be able to ascertain using no more than routine experimentation, many equivalents to the specific embodiments of the disclosure described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

What is claimed is:

1. A method for treating a tumor in a subject in need thereof, the method comprising:
administering a composition comprising antigen presenting cells to the subject, wherein
the antigen presenting cells are obtained from a species that is different than the subject.

2. The method of claim 1, wherein the tumor is a solid tumor, and the administering is into the tumor of the subject via an intratumoral injection.

3. The method of any one of the preceding claims, wherein the antigen presenting cells are obtained from a species that is a swine.

4. The method of any one of the preceding claims, wherein the antigen presenting cells are obtained from a species that is a miniature swine.

5. The method of any one of the preceding claims, wherein the subject is a human.

6. The method of claim 3, wherein the swine is an alpha-1,3 galactosyltransferase-deficient swine.

7. The method of claim 6, wherein the alpha-1,3 galactosyltransferase-deficient swine is a swine leukocyte antigen (SLA)-inbred swine.

8. The method of claim 4, wherein the miniature swine is an alpha-1,3 galactosyltransferase-deficient miniature swine.

9. The method of claim 8, wherein the alpha-1,3 galactosyltransferase-deficient miniature swine is a swine leukocyte antigen (SLA)-inbred swine.

10. The method of any one of the preceding claims, wherein the method triggers an immune response specific to the tumor.

11. The method of any one of the preceding claims, wherein the method yields an abscopal effect.

12. The method of any one of the preceding claims, wherein the tumor is a solid cancerous tumor.

13. The method of claim 12, wherein the tumor is selected from the group consisting of sarcomas, carcinomas, lymphomas, breast tumors, prostate tumors, head and neck tumors, glioblastomas, bladder tumors, pancreatic tumors, liver tumors, ovarian tumors, colorectal tumors, pulmonary tumors, cutaneous tumors, lymphoid tumors, gastrointestinal tumors, gastrointestinal stromal tumors, cervical tumors, hepatocellular carcinomas, renal cell carcinomas, melanomas, colorectal carcinomas, esophageal carcinomas, brain tumors, kidney tumors, lung tumors (including non-small cell lung cancer), gastric tumors, bile-duct tumors, uterine tumors, and childhood (pediatric) tumors.

14. The method of any one of the preceding claims, wherein the tumor is resistant to treatment to chemotherapy and/or treatment with an immunotherapy.

15. The method of any one of the preceding claims, wherein the antigen presenting cells are derived from one or more swine or miniature swine using a leukapheresis procedure, wherein the leukapheresis procedure generates a leukopak containing peripheral blood mononuclear cells, and the leukopak is further fractionated by counterflow elutriation.

16. The method of any one of the preceding claims, wherein the composition comprising antigen presenting cells is substantially free of pathogens.

17. The method of any one of the preceding claims, wherein the antigen presenting cells are obtained from swine or miniature swine of different genotypes.

18. The method of any one of the preceding claims, wherein the composition comprising antigen presenting cells is administered in single or multiple doses.

19. The method of any one of the preceding claims, wherein the composition comprising antigen presenting cells is administered via an intratumoral injection of at least about 1×10⁶ antigen presenting cells per dose.

20. The method of any one of the preceding claims, wherein the composition comprising antigen presenting cells is administered via an intratumoral injection of about 1×10⁶, about 5×10⁶, about 10×10⁶, about 15×10⁶, about 20×10⁶, about 25×10⁶, about 30×10⁶, about 35×10⁶, about 40×10⁶, about 45×10⁶, about 50×10⁶, about 55×10⁶, about 60×10⁶, about 65×10⁶, about 70×10⁶, about 75×10⁶, about 80×10⁶, about 85×10⁶, about 90×10⁶, about 95×10⁶, about 10×10⁷, about 15×10⁷, about 20×10⁷, about 25×10⁷, about 30×10⁷, about 35×10⁷, about 40×10⁷, about 45×10⁷, or about 50×10⁷ antigen presenting cells per dose.

21. The method of any one of the preceding claims, wherein the antigen presenting cells are substantially mature antigen presenting cells.

22. The method of any one of the preceding claims, wherein the antigen presenting cells are not activated or stimulated.

23. The method of any one of the preceding claims, wherein the composition comprises peripheral blood mononuclear cells (PBMC).

24. The method of any one of the preceding claims, wherein the composition comprises monocytes.

25. The method of any one of the preceding claims, wherein the composition comprises dendritic cells, macrophages, granulocytes, T-cells, B-cells, and/or NK cells.

26. The method of any one of the preceding claims, wherein the composition comprises at least about 50%, about 55%, about 60%, about 65%, about 70%, about 75%, about 80%, about 85%, about 90%, about 95%, or greater than about 95% PBMCs, wherein the PBMCs are mature, immature, or a combination of mature and immature PBMCs.

27. The method of any one of the preceding claims, wherein the composition comprises at least about 50%, about 55%, about 60%, about 65%, about 70%, about 75%, about 80%, about 85%, about 90%, about 95%, or greater than about 95% monocytes, wherein the monocytes are mature, immature, or a combination of mature and immature monocytes.

28. The method of claim 27, wherein the composition comprises a mixture of at least about 50%, about 55%, about 60%, about 65%, about 70%, about 75%, about 80%, about 85%, about 90%, about 95%, or greater than about 95% monocytes and dendritic cells, wherein the monocytes and/or dendritic cells in said mixture may be mature, immature, or a combination of mature and immature monocytes and/or dendritic cells.

29. The method of claim 28, wherein the mixture comprises from 10% to 95%, 10% to 90%, 10% to 80%, 10% to 70%, 10% to 60%, 10% to 50%, 10% to 40%, 10% to 30%, 10% to 20%, 20% to 95%, 20% to 90%, 20% to 80%, 20% to 70%, 20% to 60%, 20% to 50%, 20% to 40%, 20% to 30%, 30% to 95%, 30% to 90%, 30% to 80%, 30% to 70%, 30% to 60%, 30% to 50%, 30% to 40%, 40% to 95%, 40% to 90%, 40% to 80%, 40% to 70%, 40% to 60%, 40% to 50%, 50% to 95%, 50% to 90%, 50% to 80%, 50% to 70%, 50% to 60%, 60% to 95%, 60% to 90%, 60% to 80%, 60% to 70%, 70% to 95%, 70% to 90%, 70% to 80%, 80% to 95%, 80% to 90%, 90% to 95%, or greater than 95% monocytes.

30. The method of claim 28, wherein the mixture comprises from 10% to 30%, 35% to 55%, 60% to 80%, or 85% to 95% monocytes.

31. The method of claim 28, wherein the mixture comprises at least about 10%, about 15%, about 20%, about 25%, about 30%, about 35%, about 40%, about 45% about 50%, about 55%, about 60%, about 65%, about 70%, about 75%, about 80%, about 85%, about 90%, about 95%, or greater than about 95% monocytes.

32. The method of claim 28, wherein the mixture comprises from 10% to 95%, 10% to 90%, 10% to 80%, 10% to 70%, 10% to 60%, 10% to 50%, 10% to 40%, 10% to 30%, 10% to 20%, 20% to 95%, 20% to 90%, 20% to 80%, 20% to 70%, 20% to 60%, 20% to 50%, 20% to 40%, 20% to 30%, 30% to 95%, 30% to 90%, 30% to 80%, 30% to 70%, 30% to 60%, 30% to 50%, 30% to 40%, 40% to 95%, 40% to 90%, 40% to 80%, 40% to 70%, 40% to 60%, 40% to 50%, 50% to 95%, 50% to 90%, 50% to 80%, 50% to 70%, 50% to 60%, 60% to 95%, 60% to 90%, 60% to 80%, 60% to 70%, 70% to 95%, 70% to 90%, 70% to 80%, 80% to 95%, 80% to 90%, 90% to 95%, or greater than 95% dendritic cells.

33. The method of claim 28, wherein the mixture comprises from 10% to 30%, 35% to 55%, 60% to 80%, or 85% to 95% dendritic cells.

34. The method of claim 28, wherein the mixture comprises at least about 10%, about 15%, about 20%, about 25%, about 30%, about 35%, about 40%, about 45% about 50%, about 55%, about 60%, about 65%, about 70%, about 75%, about 80%, about 85%, about 90%, about 95%, or greater than about 95% dendritic cells.

35. The method of any one of the preceding claims, wherein the subject is receiving another anti-cancer therapy.

36. The method of claim 35, wherein the other anti-cancer therapy comprises treatment with one or more immune checkpoint inhibitors.

37. The method of claim 35, wherein the other anti-cancer therapy is an anti-CTLA4 therapy, anti-PD1 therapy, anti-PDL1 therapy, anti-LAG-3 therapy, tumor-treating fields (TTFs), cell-based therapy, a tyrosine kinase inhibitor, a VEGF inhibitor, or any combination thereof.

38. The method of claim 35, wherein the other anti-cancer therapy comprises treatment with imatinib, sunitinib, regorafenib, pazopanib, nilotinib, avapritinib, ripretinib, sorafenib, pimitespib, ipilimumab, tremelimumab, nivolumab, pembrolizumab, cemiplimab, atelizumab, avelumab, durvalumab, relatlimab, or any combination thereof.

39. The method of any one of claims 35 to 38, wherein the subject does not respond to the other anti-cancer therapy in the absence of administration of the composition comprising antigen presenting cells.

40. A pharmaceutical composition suitable for intratumoral injection, wherein the pharmaceutical composition comprises antigen presenting cells obtained from one or more swine.
